(19) United States
(12) Patent Application Publication
(10) Pub. No.: US 2025/0332434 A1
(43) Pub. Date: Oct. 30, 2025

(54) Bio-Recharge System to Normalize Body Functions

(71) Applicant: Tesla BioHealing Inc., Milford, DE (US)

(72) Inventors: James Zhou LIU, Milford, DE (US); Xianbin XU, Beijing (CN)

(21) Appl. No.: 19/199,882

(22) Filed: May 6, 2025

Related U.S. Application Data
(63) Continuation of application No. 17/897,786, filed on Aug. 29, 2022, now abandoned.
(60) Provisional application No. 63/238,138, filed on Aug. 28, 2021.

(51) Int. Cl. A61N 5/06

(52) U.S. Cl. CPC A61N 5/06; A61N 2005/0638

(57) ABSTRACT

The current invention provides a biocompatible energy recharge system to normalize body functions for a mammal. Both stationary and circulatory scalar wave energy was produced and utilized to enable the entire body of a mammal to increase the energy potential of any cell in the body, to promote cell growth, to facilitate cell repair, and to reduce cell destruction, hence the cells, organs, systems and the entire body of a mammal become healthy, and to treat a disease or disorder. The invention is a combination of the generators of the three-dimensional bio-waves in the stationary and in the circulationary formats for empowering the cells in all parts of a mammal, and a method of using the 3D bio-waves uniformly to empower each organ of a mammal to restore and maintain the normal body health and function.

### CROSS-REFERENCES TO RELATED APPLICATIONS

This application is a continuation of U.S. application Ser. No. 17/897,786, filed Aug. 29, 2022, which claims priority to U.S. Provisional Patent Application Ser. No. 63/238,138, filed Aug. 28, 2021, the contents of which are incorporated herein by reference in their entirety.

### BACKGROUND OF THE INVENTION

### 1. Field of the Invention

Being free of any disease is a universal desire. The continuation of exploring the natural or artificial resource for reaching that goal is the effort of mankind generation after generation. However, due to exposure to a variety of pathogenic factors, anybody would have aged cells/organs and get certain diseases during the lifetime. The questions were who would get which disorder/disease at which frequency. Before there is a way to have an endless lifespan, it is acceptable that the disease would be postponed as long as possible, or occur at the very late stage of life when the important cells of the body lost their cellular activities. The current invention is to provide a new bio-energy recharge system to restore cellular activities, to slow down the aging process, and to make the disease go away.

### 2. Description of Related Art

Preventing a disease from happening is the ideal medicine. Balanced nutrition is essential for maintaining normal health and body functions because nutrients are needed for every cell in the body. Vaccination is the most successful approach to prevent infectious diseases. Vaccination empowered the body against the specific infection. A healthy lifestyle and positive attitude play very important roles for a long, healthy, and happy life. Chemoprevention with a modern drug is used to prevent or postpone certain diseases. When a person or an animal is sick, there is possibly more than one type of therapy to stop the disease process. Administration of a drug is the most popular treatment of a disease, and surgery, as needed.

Effective drug can accurately target the therapeutic area. However, the principle of the drug treatment, on most occasions, was to kill a pathogen or to kill the cancer cells, or to suppress the inflammation process. Suppressing cancer cells or inflammatory reactions carries a certain degree of side effects. It is hard for a drug to be a pure benefit to a person. Therefore, many people are learning from the experience of mankind. Certain drugs, like growth hormones or hormone replacement therapy, also carries the risk of causing cancers. Energy therapy, or energy medicine, such as acupuncture to adjust the level of Qi, Qi Gong practice, are a few examples of the energy therapy used in Traditional Chinese Medicine (TCM) without noticeable long-term side effects. Far-infrared therapy, one of the modern energy medicines, has many beneficial effects when used correctly.

Although energy medicine in English was a young term first used in the 1980s, using energy to treat a disease has its unique advantages. The key character is that the applied energy was to increase the body's innate ability in self-protection and to self-fight against pathogens. This is particularly important when the body was to win the battle against any chronic disorders, such as stroke-related paralysis, terminal cancer, or post-treatment Lyme disease syndromes. The current invention is to provide a new method in the field of energy medicine to enhance self-healing.

Back in 400 B.C., the great Greek physician Hippocrates of Kos told people that everyone had an innate healing system. It is still very important that the innate healing system should be enhanced and utilized for maintaining normal health. Optimum health heavily relies on the innate healing capacity of the body. The current invention is to enhance the body's innate healing ability.

A well-known physician and pharmacist in the history of the Traditional Chinese Medicine (TCM) named Li Shizhen told people that “for body healing, food was better than a drug; water was better than food, sleep was better than water.” Therefore, the importance of a good night's sleep for maintaining health and normal body function was recognized a very long time ago.

Very recently, on Nov. 1, 2019, a group of scientists at Boston University and Harvard University reported that only during deep sleep time, the brain can effectively remove the metabolic wastes—such as amyloid-beta. If the metabolic wastes were accumulated, the cognition capacity and brain tissue health would be in question, which could be a risk factor for developing Alzheimer's disease (AD). AD is an irreversible, progressive brain disorder that slowly destroys memory and thinking skills and, eventually, the ability to carry out the simplest tasks. So far, there is no effective therapy for AD.

There are many methods to promote deep sleep. Dietary supplements, such as melatonin, has been used for years to enhance sleep. Many drugs, either prescription drugs or over the counter drugs, are available for promoting sleep. Acupuncture or many other types of Traditional Chinese Medicine was used to promote sleep. All of these therapies were aimed at the brain in a hope that after the brain is energized, the whole body could be empowered. However, it was only partially true. For reaching optimum health, the whole body, every part of the body, every cell in the body, needs to be fully recharged with energy. This is a part of the new field of energy medicine.

Compared to drug-based internal medicine, the term energy medicine was relatively new, although the history of energy medicine has a history of several thousands of years. Many approaches to energy medicine were applied to energize the body, such as dietary supplements, drug therapies, physical therapy, far infrared therapy, etc. There are numerous prior arts. However, the scalar energy offered a very different mechanism of action in energizing the cells.

Several scalar-wave based devices have been used to treat a variety of diseases. The commonly used one type was the scalar wave laser. More than 50 types of diseases were successfully treated with scalar wave laser, such as asthma, allergy, sleep disorder, Alzheimer's disease, high blood pressure, stress, etc. Typing in “scalar wave laser” yielded 1.6 million entries. However, performing any laser to achieve the actual therapeutic effect needs a therapist with a special skill. The example was that the acupuncture needles can be used to treat a variety of diseases/disorders, even for anesthetizing the patient during surgery. However, using the needle requires a formerly training either in school or by a long-term coach. If a patient can use a device for self-healing, such as a therapeutic bed, that will be very welcome.

In the current market, a scalar wave device called Quantum Resonance Crystal Bed is used. It is a combination of crystals and light working together to heal the body. The transmitted energy cleanses, energizes, and balances the chakras or energy centers in the human body. It was reported that this Quantum Resonance Crystal Bed brings a deep sense of peace; reduces stress and anxiety, restores the body and boosts energy, balances the energy system inside the body, provides deeper, restive sleep, and more. Because the treatments are non-invasive and easy to perform, they are suitable for all ages. Those who benefit the most are people who are encountering stress, not feeling energetic, or those who want to clear their minds and start anew. For those who try the crystal bed sessions complete with quantum resonance, the results have been quite noticeable. However, the Quantum Resonance Crystal Bed was not used as a typical bed. The seven crystal lights must focus on the 7 chakras to be effective.

One of the devices used in Quantum Medicine is Quantum Resonance System (QRS). The QRS is to enhance cell preservation and cell regeneration. It can be applied to diseases and physical maladies. The QRS home system can be operated comfortably at home. It was reported that the QRS transports hydrogen to accomplish cellular entry of calcium, sodium, potassium, chlorine, and magnesium ions. Thus, it allows for better cell supply and freed the same cell of waste products and toxins. The QRS significantly increased oxygen in the tissues with the oxygen diffusion rate increased by 80 to 900%, and the energy molecules (ATP) were formed. This allows the QRS system to transport the materials through solidified or slackened tissue structures. The QRS, however, only provides the stationary scalar energy to the human body. Thus, the body would not be uniformly energized from all directions. It would be more desirable that the circulatory energy is an important part to let the whole body to receive the energy from all directions, not only from the blanket-like device the person laid on.

Spooky2 Scalar acts slightly differently from QRS. It has both a sender and a receiver of scalar-wave. The sender is tuned to the receiver so they are perfectly linked. There is only a single wire between the two. A scalar field is only produced when the two units are tuned. They become charge-coupled, effectively becoming one entity; one transmitting a regular electrical signal, and the other returning the signals in the form of a scalar field. The lids become a transmitting antenna of scalar energy, with a scalar field between them. The return path is the space between the lids. The scalar field goes in all directions, but a receiver will absorb this energy, in a similar way to the ionized gas in a plasma ball. The finger absorbs the energy, and a stream of electrons passes through the glass to the finger. When this happens, the energy becomes more focused. The person being treated sits between the sender and receiver, and they also become a tuned receiver. Spooky scalar is another device to broadcast scalar waves in a stationary manner, not in a circulatory manner. Therefore, the whole body would not be uniformly energized from all directions. Rather, the energy waves enter into the body from one side and leave the body from the other side.

It would be more desirable that the circulatory energy is an add-on for the Spooky system to let the whole body to receive the energy from all directions, not only from one side go through the other side of the body.

The current invention offers both stationary and circulatory scalar wave energy around the entire body to increase the cellular repair rate, to decrease cell destruction rate, to promote the cell metabolism, to detoxify toxins or free radicals, to remove waste products from the cells, and bio-recharge cells to the full voltage level. All cells and all organs in the whole body become energized.

### SUMMARY OF THE INVENTION

The objective of the present invention is to recharge a mammal's body to make the cells in any part of the body to be fully energized. Then, the health and body function of a mammal would be back to an ideal status. The invention incorporates the scalar wave energy, vacuum zero-point energy, and scalar wave energy into one device that can broadcast a bio-healing energy field in a stationary and a circulatory format, which can facilitate the energy recharge of the cells in any parts of the body uniformly. Utilizing this bio-healing energy field, the inventors have helped more than 600 patients to improve their health conditions significantly and treated many important diseases, such as paralysis after stroke, terminal cancers, hypertension, diabetes, etc. These people with chronic fatigue were recharged and their daily lives were full of energy. Several patients corrected their misshape (disfigure) due to severe osteoporosis. Many patients with insomnia changed to have a long-time waited for normal sleep. The depressed patients became normal individuals and were happy. The patients with mental stress were relieved. The elderly patients improved their life quality due to free of life without this and that physical restriction. Many children resumed normal growth and their mental development was improved.

In accordance with embodiments of the invention, a biocompatible scalar wave energy generation system to increase stem cell production, to promote cell growth, to facilitate cell repair, to reduce cell damage, to improve health, and to treat a disease of a mammal is proved. The system comprises (1) a stationary scalar wave energy field generator, comprising a bracket and a supporting frame system, a scalar wave energy field source component, an electricity power receiver, a scalar wave energy field conduction system, a scalar wave energy field flow-guide controller, a scalar wave energy field collector, and a scalar wave energy field release device to enable the scalar wave energy to flow to a mammal in a stationary manner; or (2) a circulatory scalar wave energy filed generator, comprising a bracket and a supporting frame system, a scalar wave energy field source component, an electricity power receiver, a scalar wave energy field conduction system, a scalar wave energy field flow-guide controller, a scalar wave energy field collector, and a scalar wave energy field release device to enable the scalar wave energy to flow around to a mammal in a circulatory manner; or (3) a stationary or a circulatory scalar wave energy filed generator, comprising a bracket and a supporting frame system, a scalar wave energy field source component, a scalar wave energy field conduction system, a scalar wave energy field flow-guide controller, a scalar wave energy field collector, and a scalar wave energy field release device to enable the scalar wave energy to flow around to a mammal in a stationary or a circulatory manner; and (4) a rest apparatus, like a bed, or a pad, or a chair, empowered with scalar wave energy to enable a mammal to receive stationary and/or circulatory scalar wave energy so the entire body of a mammal can increase the bioenergy potential.

In accordance with embodiments of the invention, a process of using a therapeutic device generating and releasing bioenergy from a stationary scalar wave energy field generator or a circulatory scalar wave energy filed generator, to enable the entire body of a mammal to increase the energy potential, to promote stem cell production, to promote cell growth, to facilitate cell repair, and to reduce cell destruction, to cause the cells, organs, systems and the entire body of a mammal become healthy, and to treat a disease or a disorder of a mammal is provided.

In accordance with embodiments of the invention, a method of using a device generating and releasing bioenergy from a stationary scalar wave energy field generator or a circulatory scalar wave energy filed generator, to enable the entire body of a mammal to increase the energy potential, to promote stem cell production, to promote cell growth, to facilitate cell repair, and to reduce cell destruction, to cause the cells, organs, systems and the entire body of a mammal become healthy, and to treat a disease or a disorder of a mammal is provided.

### DRAWINGS

FIG. 1. Support frame for a scalar wave generator, or a bio-recharge energy source. The multiple layers of the frame are to fix the conducting wires.

FIG. 2. A support frame for the assembly of one layer of the scalar wave generator. Many holes were needed to fix the components of the scalar wave generator.

FIG. 3. A support frame for the assembly of one layer of scalar wave generator.

FIG. 4. The individual units of the scalar wave generator to provide the bio-recharge energy.

FIG. 5. A scalar wave transporter and collector.

FIG. 6. A scalar wave transporter and releaser.

FIG. 7. A curtain-like circulator for scalar wave circulation.

FIG. 8. A healing-space (bed) empowered with a bio-recharge energy source, which is a scalar wave energy field supplied with stationary and circulatory scalar waves.

FIG. 9. An image of the micromagnetic resonance imaging (mMRI) scan showing the improved cellular activities in the brain after the man was treated inside of the scalar-wave-powered healing bed for three nights as compared to the baseline condition.

FIG. 10. An image of the micromagnetic resonance imaging (mMRI) scan showing the improved cellular activities of epiphysis after the man was treated inside of the scalar wave-powered healing bed for three nights as compared to the baseline condition.

FIG. 11. An image of the micromagnetic resonance imaging (mMRI) scan showing the improved cellular activities of the thyroid and parathyroid glands after the man was treated inside of the scalar wave-powered healing bed for three nights as compared to the baseline condition.

FIG. 12. An image of the micromagnetic resonance imaging (mMRI) scan showing the improved cellular activities of the right artery of the head and neck after the man was treated inside of the scalar wave-powered healing bed for three nights as compared to the baseline condition.

FIG. 13. An image of the micromagnetic resonance imaging (mMRI) scan showing the improved cellular activities of the right shoulder joint after the man was treated inside of the scalar wave-powered healing bed for three nights as compared to the baseline condition.

FIG. 14. An image of the micromagnetic resonance imaging (mMRI) scan showing the improved cellular activities of the left knee joint after the man was treated inside of the scalar wave-powered healing bed for three nights as compared to the baseline condition.

FIG. 15. An image of the micromagnetic resonance imaging (mMRI) scan showing the improved cellular activities of the lung (trachea and bronchi) after the man was treated inside of the scalar wave-powered healing bed for three nights as compared to the baseline condition.

FIG. 16. An image of the micromagnetic resonance imaging (mMRI) scan showing the improved cellular activities of the heart-blood vessels after the man was treated inside of the scalar wave-powered healing bed for three nights as compared to the baseline condition.

FIG. 17. An image of the micromagnetic resonance imaging (mMRI) scan showing the improved cellular activities of main blood vessels after the man was treated inside of the scalar wave-powered healing bed for three nights as compared to the baseline condition.

FIG. 18. An image of the micromagnetic resonance imaging (mMRI) scan showing the improved cellular activities of the thymus after the man was treated inside of the scalar wave-powered healing bed for three nights as compared to the baseline condition.

FIG. 19. An image of the micromagnetic resonance imaging (mMRI) scan showing the improved cellular activities of the liver after the man was treated inside of the scalar wave-powered healing bed for three nights as compared to the baseline condition.

FIG. 20. An image of the micromagnetic resonance imaging (mMRI) scan showing the improved cellular activities of liver-cells after the man was treated inside of the scalar wave-powered healing bed for three nights as compared to the baseline condition.

FIG. 21. An image of the micromagnetic resonance imaging (mMRI) scan showing the improved cellular activities of adrenals after the man was treated inside of the scalar wave-powered healing bed for three nights as compared to the baseline condition.

FIG. 22. An image of the micromagnetic resonance imaging (mMRI) scan showing the improved cellular activities of the pancreas after the man was treated inside of the scalar wave-powered healing bed for three nights as compared to the baseline condition.

FIG. 23. An image of the micromagnetic resonance imaging (mMRI) scan showing the improved cellular activities of the intestine after the man was treated inside of the scalar wave-powered healing bed for three nights as compared to the baseline condition.

FIG. 24. An image of the micromagnetic resonance imaging (mMRI) scan showing the improved cellular activities of the kidney after the man was treated inside of the scalar wave-powered healing bed for three nights as compared to the baseline condition.

FIG. 25. An image of the micromagnetic resonance imaging (mMRI) scan showing the improved cellular activities of the pelvis cavity after the man was treated inside of the scalar wave-powered healing bed for three nights as compared to the baseline condition.

FIG. 26. An image of the micromagnetic resonance imaging (mMRI) scan showing the improved cellular activities of the male reproduction system after the man was treated inside of the scalar wave-powered healing bed for three nights as compared to the baseline condition.

FIG. 27. An image of the micromagnetic resonance imaging (mMRI) scan showing the improved cellular activities of the knee joint after the man was treated inside of the scalar wave-powered healing bed for three nights as compared to the baseline condition.

FIG. 28. An image of the micromagnetic resonance imaging (mMRI) scan showing the improved cellular activities of the left foot after the man was treated inside of the scalar wave-powered healing bed for three nights as compared to the baseline condition.

FIG. 29. An image of the micromagnetic resonance imaging (mMRI) scan showing the improved cellular activities of red bone marrow after the man was treated inside of the scalar wave-powered healing bed for three nights as compared to the baseline condition.

FIG. 30. An image of the micromagnetic resonance imaging (mMRI) scan showing the improved cellular activities of blood cells after the man was treated inside of the scalar wave-powered healing bed for three nights as compared to the baseline condition.

FIG. 31. An image of the micromagnetic resonance imaging (mMRI) scan showing the improved cellular activities of thrombocyte after the man was treated inside of the scalar wave-powered healing bed for three nights as compared to the baseline condition.

FIG. 32. An image of the micromagnetic resonance imaging (mMRI) scan showing the improved cellular activities of digestive glands after the man was treated inside of the scalar wave-powered healing bed for three nights as compared to the baseline condition.

FIG. 33. An image of the micromagnetic resonance imaging (mMRI) scan showing the improved cellular activities of lymphatic vessels after the man was treated inside of the scalar wave-powered healing bed for three nights as compared to the baseline condition.

FIG. 34. An image of the five-character-scanning of a Traditional Chinese Medicine showing the improved five-characters after the man was treated inside of the scalar wave-powered healing bed for three nights as compared to the baseline condition.

FIG. 35. An image of the five-character-scanning of a Traditional Chinese Medicine showing the improved Yin-Yang balance after the man was treated inside of the scalar wave-powered healing bed for three nights as compared to the baseline condition.

FIG. 36. An image of the micromagnetic resonance imaging (mMRI) scan showing the improved brain cellular activities of the cerebrum (top view) after the stroke-paralyzed patient was treated inside of the scalar wave-powered healing bed for six days-nights as compared to the baseline condition.

FIG. 37. An image of the micromagnetic resonance imaging (mMRI) scan showing the improved brain cellular activities of the cerebrum (cross sectional view) after the stroke- paralyzed patient was treated inside of the scalar wave-powered healing bed for six days-nights as compared to the baseline condition.

FIG. 38. An image of the micromagnetic resonance imaging (mMRI) scan showing the improved brain cellular activities of nuclei of cranial nerves (back view) after the stroke-paralyzed patient was treated inside of the scalar wave-powered healing bed for six days-nights as compared to the baseline condition.

FIG. 39. An image of the micromagnetic resonance imaging (mMRI) scan showing the improved cellular activities of nerves of the back after the stroke-paralyzed patient was treated inside of the scalar wave-powered healing bed for six days-nights as compared to the baseline condition.

FIG. 40. An image of the micromagnetic resonance imaging (mMRI) scan showing the improved cellular activities of the hand after the stroke-paralyzed patient was treated inside of the scalar wave-powered healing bed for six days-nights as compared to the baseline condition.

### DETAILED DESCRIPTION OF THE INVENTION

The present invention is a spin-wave bio-recharge energy system which is to empower the body's cells for promoting the health and function of the body. This system is described in detail below regarding the accompanying drawings. In biology, the bio-recharge energy system can also be called “a quantum cell activator.”

The bio-recharge energy system disclosed in the present invention comprises at least one scalar wave generator, a scalar wave field guide-controller, a scalar wave field collector, a scalar wave field releaser; and energy formed space in which a mammal can rest and/or sleep.

### A Scalar Wave Generator

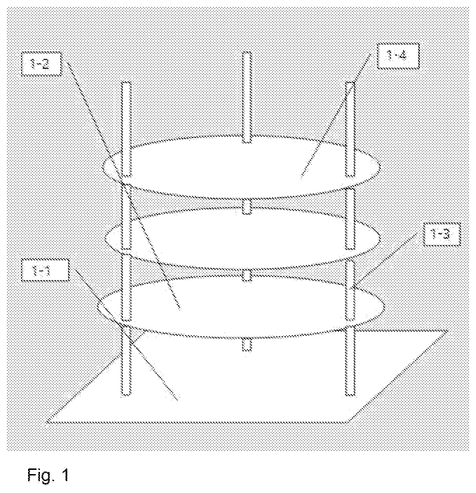

As shown in FIG. 1, the scalar wave generator supporting frame (1) includes a bottom plate (1-1), a bracket (1-2) (not shown here but will show in FIG. 3), three columns (1-3), and three circular support frames (1-4) which covered the brackets (1-2). Three columns (1-3) are equidistant. One end of the column is fixed on the bottom plate (1-1) to form an equilateral triangle. Three support frames (1-4) are fixed to the column (1-3) in parallel with each other, and the middle of the support frame (1-4) is adjacent to the upper and lower circular supports. The distances of the frames (1-4) are equal. Therefore, the bottom (1-1), the columns (1-3), and the plates (1-4) form a strong support frame for the scalar wave generator.

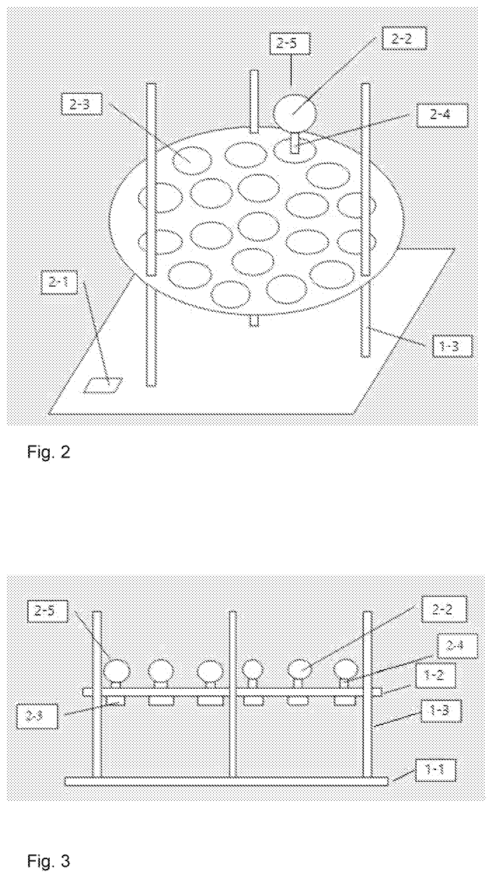

As shown in FIG. 2, a scalar wave generator assembly includes a DC power source (2-1); the body of the scalar wave generator (2-2); and a brushless motor (2-3). Each of the brushless motors respectively connected to the body of the scalar wave generator (2-2). The body of the scalar wave generator (2-2) has a shell (2-5), which contains the scalar wave-generating source material (2-2) and a tubular connector (2-4) to connect with the brushless motor (2-3). The scalar wave-generating source material can be perovskite, ferromagnetic insulator yttrium iron garnet, MnAs ferromagnetic nanoparticles, etc. The body of the scalar wave generator (2-2), the brushless motor (2-3), and the tubular connector (2-4) are to form a unit. Each unit is uniformly fixed in one of 19 holes of the supporting frame to form the scalar wave generator assembly.

As shown in FIG. 3, the side view of the partially assembled scalar wave generator includes a ball-like source-material of scalar wave generator (2-2), a DC power source (2-1); and a brushless motor (2-3). The brushless motor (2-3) respectively connected to the ball-like source-material of scalar wave generator (2-2) and the DC power source (2-1). The ball-like source-material of the scalar wave generator (2-2) has a shell (2-5) to contain the source-material connected to the brushless motor (2-3).

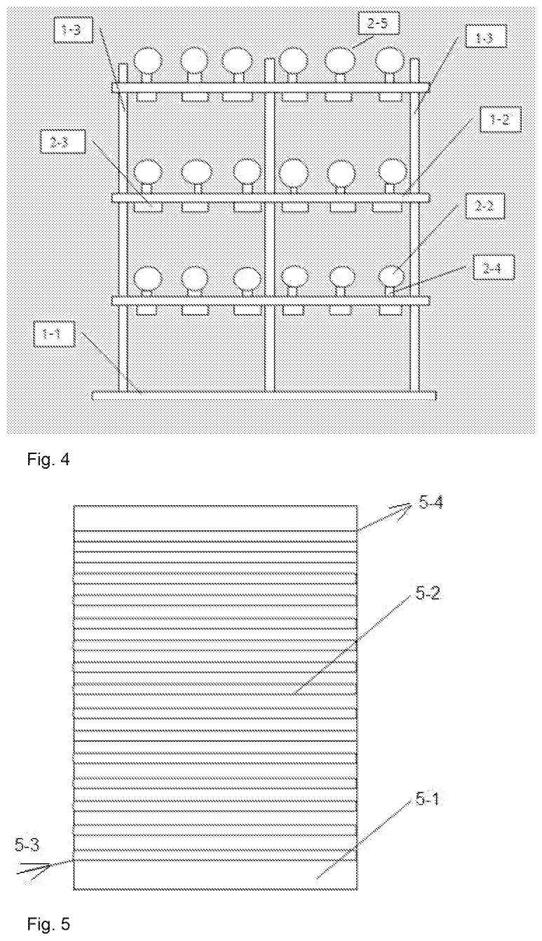

As shown in FIG. 4, the side view of the fully assembled scalar wave generator includes a ball-like source-material of scalar wave generator (2-2), a DC power source (2-1); a brushless motor (2-3), a tubular connector (2-4). All those parts are fixed into the supporting frames supported by the bottom-base (1-1), the brackets (1-2), the columns (1-3), and the support frame (1-4). After the DC power is switched on, the scalar wave generator assembly will produce the scalar wave.

Further, the shell (2-5) is spherical, the brushless motors (2-3) are parallel brushless motors, the power source (2-1) is a DC power source, and the length and diameter of the tubular (2-4) connecting member are matched with the output shaft of the brushless motor. The support frame is plate-shaped (1-4).

Further, the number of the plate-shaped support frames is three columns and is parallel to each other, and a vertical hole on which the brushless motor is mounted is evenly disposed of.

Further, the brushless motor and the casing accommodating the scalar wave field source material are respectively located on the upper and lower sides of the plate-shaped support frame.

Further, there are between 5.0 and 15 grams of said scalar wave field source material in each of said shells. The number of said scalar wave field source components on each of said support frames is 19+/−10. Preferably, the scalar wave field source assembly comprises a scalar wave field source and a brushless motor; the brushless motor is respectively connected to the scalar wave field source body and the DC power source; the field source body comprises a field source material and a shell of the field source material. And a tubular connector is connected to the brushless motor.

### The Scalar Wave Filed Collector

The scalar wave field collector provided by the present invention shown in FIG. 5 is described in detail below. The body of the scalar wave field collector is composed of a support cylinder (5-1) and a spiral wire (5-2). The wire is a copper enamel wire with a diameter of about 0.77 mm, which is wounded clockwise from the top to the bottom of the support cylinder. The lower end of the copper enameled wire spiral (5-3) is the field body inlet end of the scalar wave field body collector, and the upper end of the copper enameled wire (5-4) is a scalar wave field outlet end of the scalar wave field collector. The inner diameter of the scalar wave field collector is larger than the outer diameter of the scalar wave field generator shown in FIG. 1-4 to place the scalar wave field generator inside of the body of the scalar wave field collector shown in FIG. 5.

### The Scalar Wave Field Releaser

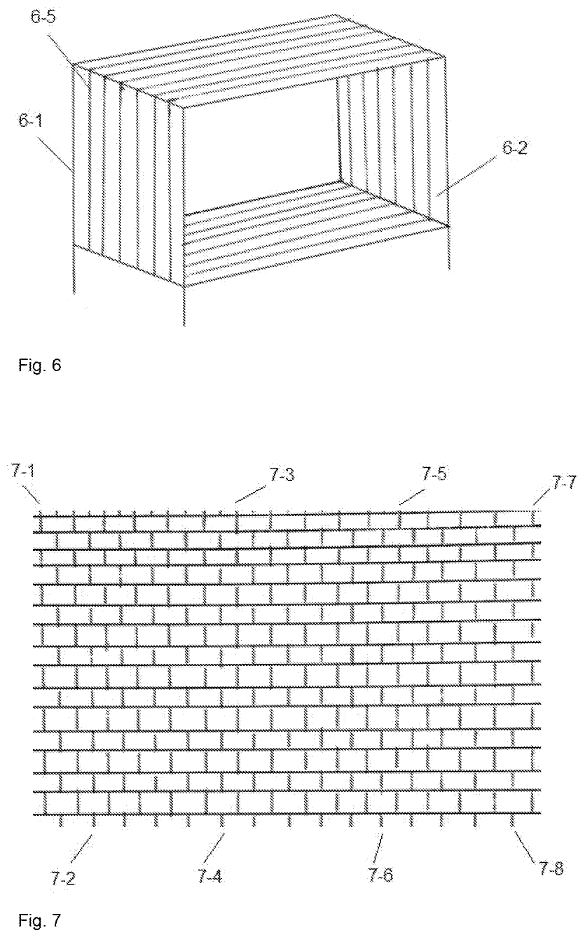

The scalar wave field release device shown in FIG. 6 is described in detail below. The scalar wave field releaser comprises a support frame (6-1) and a cylindrical member (6-2). The cylindrical member releaser opens at both ends. The cylindrical member (6-2) is covered by an oxygen-free pure copper enameled wire (6-3), which is spirally woven on the strip-shaped support members (6-4) when it was placed in parallel. When the cylindrical member (6-2) is horizontally placed, the left-to-right spiral (6-5) is clockwise. The left end of the spiral line (6-5) is the entrance end of the scalar wave field, which is connected to the exit end of the scalar wave field collector. The right end of the spiral line (6-5) is the exit end of the scalar wave field, and the scalar wave field input terminals of the field guide controller (3) are connected. In use, the tubular member (6-2) surrounds the user's body, such as a soft cloth, or a bed, and is mounted as a rectangular cylinder of up, down, top, bottom, or left and right. The one side (end) of the bed is for the user to enter into and exit the bed.

The curtain-like circulator for scalar wave circulation as shown in FIG. 7. The scalar wave circulation comprises a support frame and multiple networks of the scalar wave energy flow-through. The network 7-1 and 7-2, 7-3 and 7-4, 7-5 and 7-6, 7-7 and 7-8, and more, are independently forming a wave-flows. Inside of the framed many wave-flows, scalar wave energy is circulated day and night so the user can constantly be surrounded by the therapeutic energy.

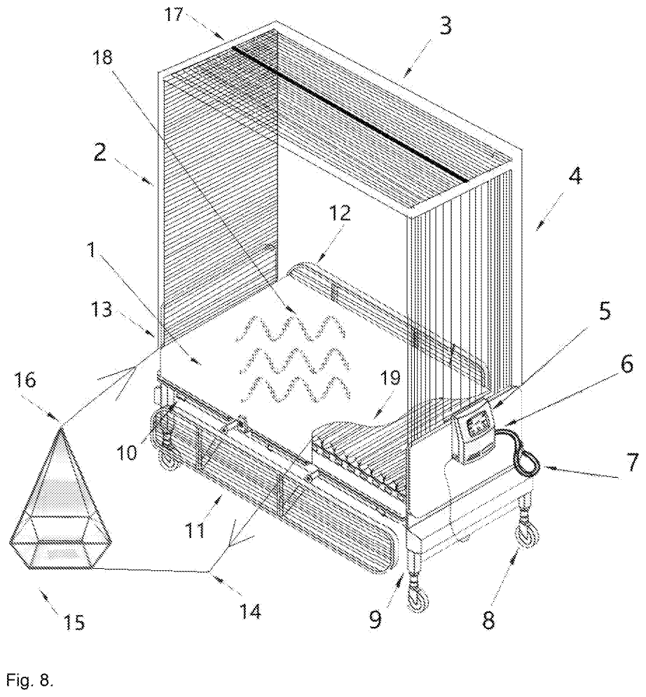

FIG. 8 is an engineering drawing that shows a scalar wave-powered one zone healing bed (1Zone Bed) for the patient to lie on. The label of FIG. 8:1—Mattress including the healing pad empowered with scalar wave energy, the bottom of the 1—Zone Bed; 2—Headboard side of the 1—Zone Bed; 3—top side of the 1—Zone Bed; 4—Footboard side of the 1—Zone Bed; 5—Control Switch; 6—Cable; 7—Frame Back Section; 8—Wheel; 9—Frame Front Section; 10—Fixer of the Rail; 11—Front Rail; 12-Back Rail; 13—Entry Wire of the Circular Scalar Wave Energy; 14—Out Wire of the Circular Scalar Wave Energy; 15—Scalar Wave Energy Source; 16—Scalar Wave Energy Source output; 17—Hanger Rod Support for Hanging an IV-Fluid or a Tube Feeding; 18. Scalar Wave Energy field inside of the 1Zone Healing Bed.

The sleeping space of the 1—Zone Scalar Wave Energy Healing Bed is formed with a mattress including the healing pad and the headboard (Part 2), footboard (Part 4), the top (Part 3), the left (Part 11) and the right sides (Part 12) with the supporting frame. The side-area 1, 2, 3 and 4 were formed by using one large piece of the foldable sheet along with a 4-corner supported frame. The left or right side was formed with a whole piece of Part 11 or Part 12. Part 11 or Part 12 has the same full-length as the mattress so when it is raised up, there would be no extra zone formed between the rail and the headboard, or between the rail and the footboard. After Part 11 or Part 12 is raised up, only 1-Zone space is formed. Part 11 or Part 12 itself has no zone. Part 11 or Part 12 can be adjusted to a higher or lower position or lowered down to the same level of the upper surface of the mattress depending on the actual need for patient care. Both headboard and footboard have no extra zone to form other than the 1-zone, so it will not cause entrapment. There is no any other zone is created inside of the smooth 1-zone space. This can almost eliminate any risk of entrapment or falls. This is a safe and effective healing bed for recharging a patient whenever the patient is sitting or lying on the bed. This is especially advantageous for use by the patient with a chronic disease or the body is very weak.

Further, the scalar wave energy powered healing bed was tested by a man who practiced Qi Gong and was especially sensitive to the biocompatible scalar wave energy. When he was lying on the bed, he could feel special energy around him. When the plug-in electricity was pulled off, he did not feel any special energy around him. Also, his Qi Gong practice had a high speed of improvement when he did practice inside of the scalar wave energy powered healing bed. He also could feel the uncomfortableness if the scalar wave energy was produced by the typical Tesla coil. Therefore, the current invention is a modified Tesla coil and both stationary and circulatory scalar wave energy were generated to enable the entire body of a mammal to increase the energy potential of any cell in the body, to promote cell growth, to facilitate cell repair, and to reduce cell destruction, hence the cells, organs, systems and the entire body of a mammal become healthy, and to treat a disease or disorder, as shown in below images.

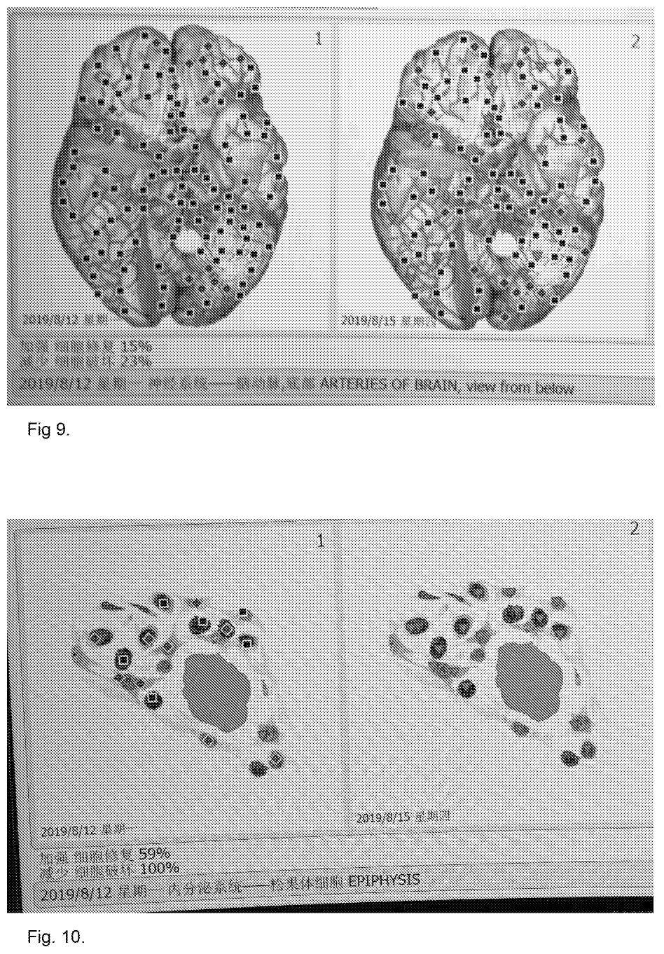

FIG. 9 is an image of the micromagnetic resonance imaging (mMRI) scan showing the improved cellular activities of the brain arteries after the 63-year old man was treated inside of the scalar wave-powered healing bed for three nights as compared to the baseline condition. The cellular repair rate of the brain arteries was increased by 15%, and the cellular destruction rate of the brain arteries was reduced by 23%. This result clearly indicated that the scalar wave treatment for three nights significantly improved the cellular activity of the brain arteries. The improved brain arteries can supply the brain nerve tissue with constant blood, so the brain cells can be surely nourished with nutrients and oxygen. Many sub-organs inside of the brain, such as a visual cortex of the brain, could have a better function, or improved vision.

FIG. 10 is an image of the micromagnetic resonance imaging (mMRI) scan showing the improved cellular activities of epiphysis after the 63-year old man was treated inside of the scalar wave-powered healing bed for three nights as compared to the baseline condition. The cellular repair rate of epiphysis inside of the brain was increased by 59%, and the cellular destruction rate of the epiphysis was reduced by 100%. This result clearly indicated that the scalar wave treatment for three nights significantly improved the cellular activity of the epiphysis, the pineal gland. The improved epiphysis can produce more melatonin, which then helps the patient to improve the quality of sleep. As a consequence, overall health was improved. The treatment inside of the biocompatible scalar wave energy powered healing bed improved the sleep quality of the patient. The patient reported that his brainpower became much better than before, and had less tiredness during the long-writing hours.

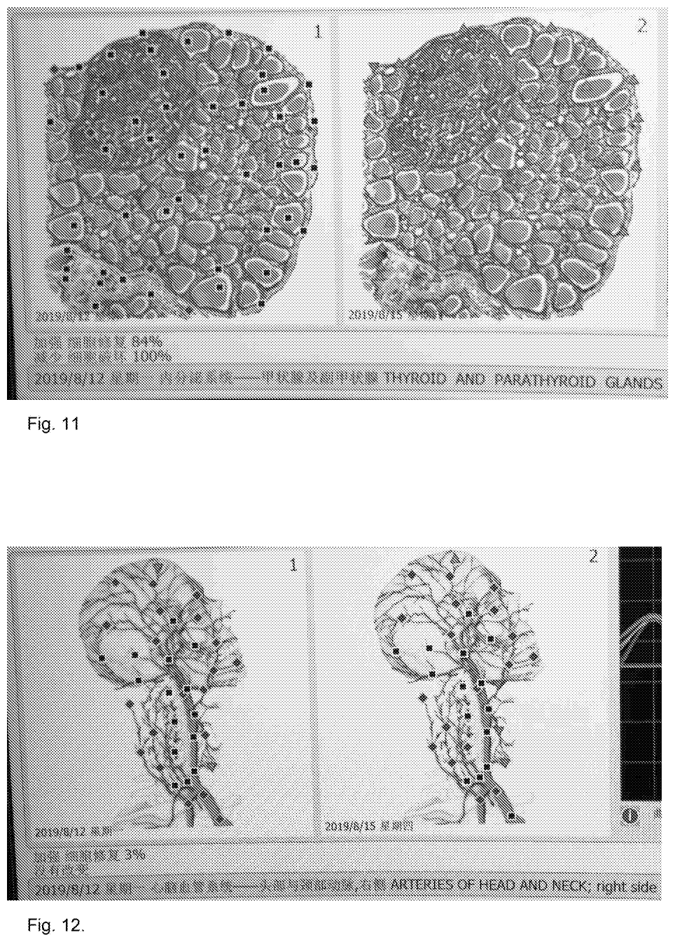

FIG. 11 is an image of the micromagnetic resonance imaging (mMRI) scan showing the improved cellular activities of the thyroid and parathyroid glands after the 63-year old man was treated inside of the scalar wave-powered healing bed for three nights as compared to the baseline condition. The cellular repair rate of the thyroid and parathyroid glands was increased by 84%, and the cellular destruction rate of the thyroid and parathyroid glands was reduced by 100%. This result clearly indicated that the scalar wave treatment for three nights significantly improved the cellular activity of the thyroid and parathyroid glands. The improved function of the thyroid and parathyroid glands can regulate the body to have many health benefits.

FIG. 12 is an image of the micromagnetic resonance imaging (mMRI) scan showing the improved cellular activities of the right artery of the head and neck after the 63-year old man was treated inside of the scalar wave-powered healing bed for three nights as compared to the baseline condition. The cellular repair rate of the head and neck was increased by 3%, and the cellular destruction rate of the right artery of the head and neck was unchanged. This result indicated that the scalar wave treatment for three nights might not be long enough to improve the cellular activity of the right artery of the head and neck.

FIG. 13 is an image of the micromagnetic resonance imaging (mMRI) scan showing the improved cellular activities of the right shoulder joint after the 63-year old man was treated inside of the scalar wave-powered healing bed for three nights as compared to the baseline condition. The cellular repair rate of the right shoulder joint was increased by 45%, and the cellular destruction rate of the right shoulder joint was reduced by 100%. This result clearly indicated that the scalar wave treatment for three nights significantly improved the cellular activity of the right shoulder joint.

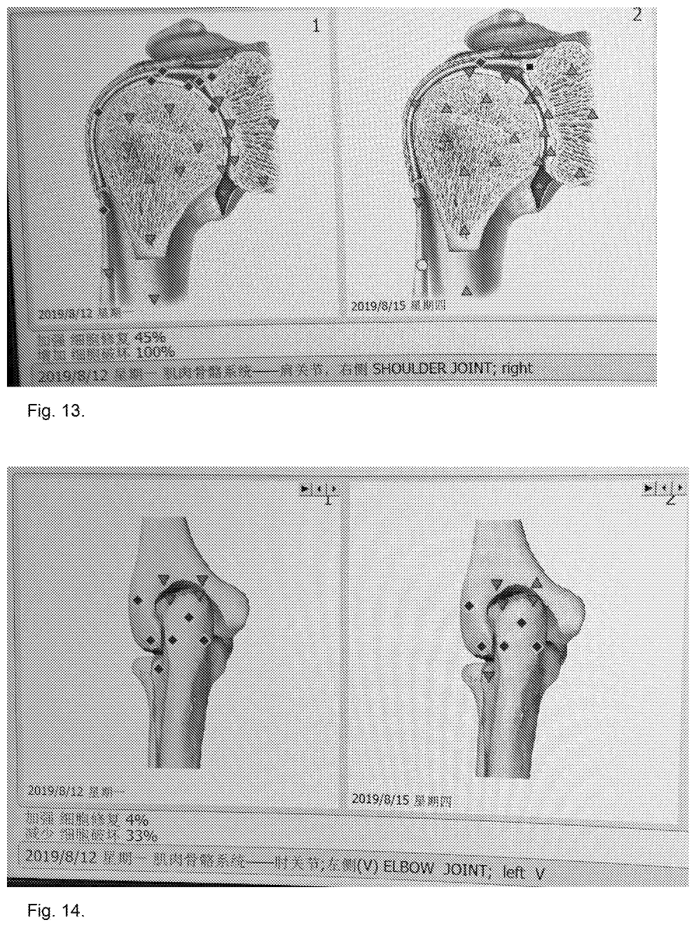

FIG. 14 is an image of the micromagnetic resonance imaging (mMRI) scan showing the improved cellular activities of the left elbow joint after the 63-year old man was treated inside of the scalar wave-powered healing bed for three nights as compared to the baseline condition. The cellular repair rate of the left elbow joint was increased by 45%, and the cellular destruction rate of the right shoulder joint was reduced by 100%. This result clearly indicated that the scalar wave treatment for three nights significantly improved the cellular activity of the left elbow joint.

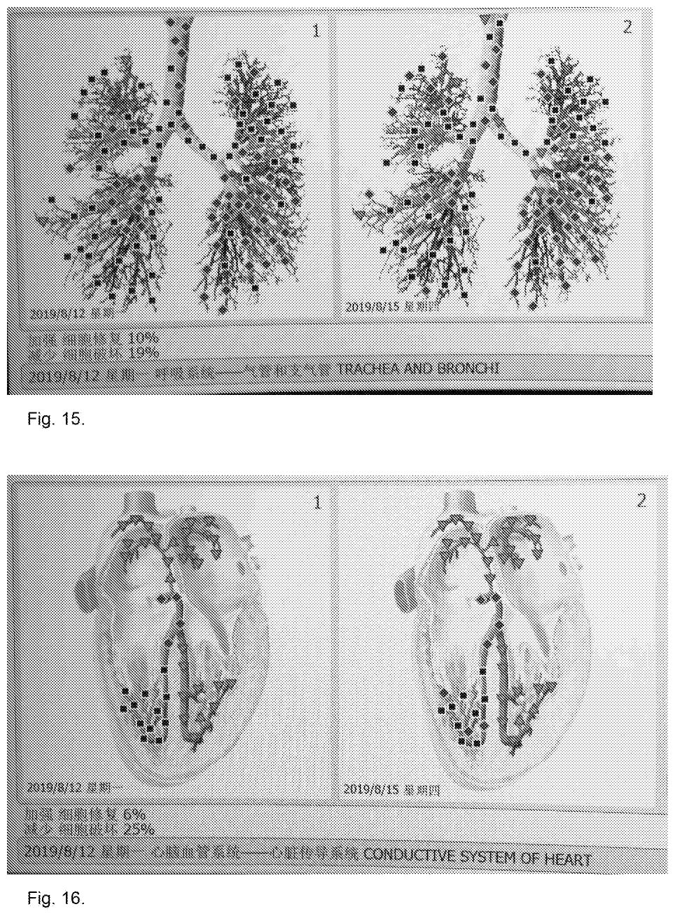

FIG. 15 is an image of the micromagnetic resonance imaging (mMRI) scan showing the improved cellular activities of the lung (trachea and bronchi) after the 63-year old man was treated inside of the scalar wave-powered healing bed for three nights as compared to the baseline condition. The cellular repair rate of the lung (trachea and bronchi) was increased by 10%, and the cellular destruction rate of the brain arteries was reduced by 19%. This result clearly indicated that the scalar wave treatment for three nights significantly improved the cellular activity of the lung, the respiratory trees.

FIG. 16 is an image of the micromagnetic resonance imaging (mMRI) scan showing the improved cellular activities of the heart-blood vessels after the 63-year old man was treated inside of the scalar wave-powered healing bed for three nights as compared to the baseline condition. The cellular repair rate of the heart-blood vessels was increased by 6%, and the cellular destruction rate of the brain arteries was reduced by 25%. This result clearly indicated that the scalar wave treatment for three nights significantly improved the cellular activity of the heart-blood vessels. After the heart-blood vessels were improved, the function of the heart would be improved so does the overall blood supply to the whole body.

FIG. 17 is an image of the micro-magnetic resonance imaging (mMRI) scan showing the improved cellular activities of the main blood vessels after the 63-year old man was treated inside of the scalar wave-powered healing bed for three nights as compared to the baseline condition. The cellular repair rate of the main blood vessels was increased by 9%, and the cellular destruction rate of the brain arteries was reduced by 17%. This result clearly indicated that the scalar wave treatment for three nights significantly improved the cellular activity of the main blood vessels. The related health benefit will be to reduce the risk of cardiovascular diseases.

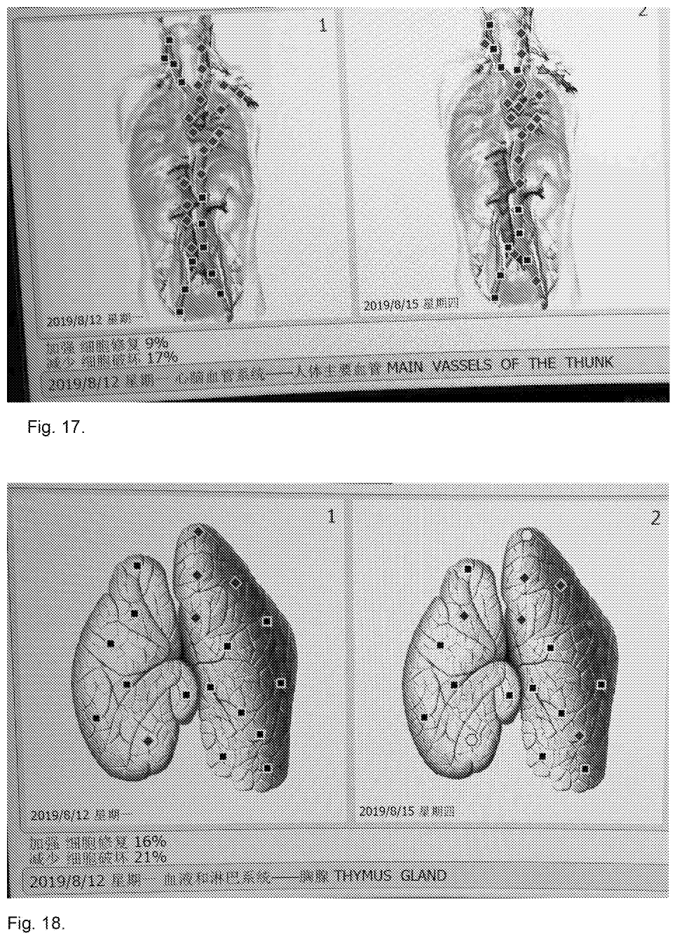

FIG. 18 is an image of the micro-magnetic resonance imaging (mMRI) scan showing the improved cellular activities of the thymus after the 63-year old man was treated inside of the scalar wave-powered healing bed for three nights as compared to the baseline condition. The cellular repair rate of the thymus was increased by 16%, and the cellular destruction rate of the thymus was reduced by 21%. This result clearly indicated that the scalar wave treatment for three nights significantly improved the cellular activity of the thymus. The thymus is the critical organ to make the t-lymphocytes which play a critical role in defending the body against microbial pathogens and cancer development. Increased thymus function has numerous health benefits.

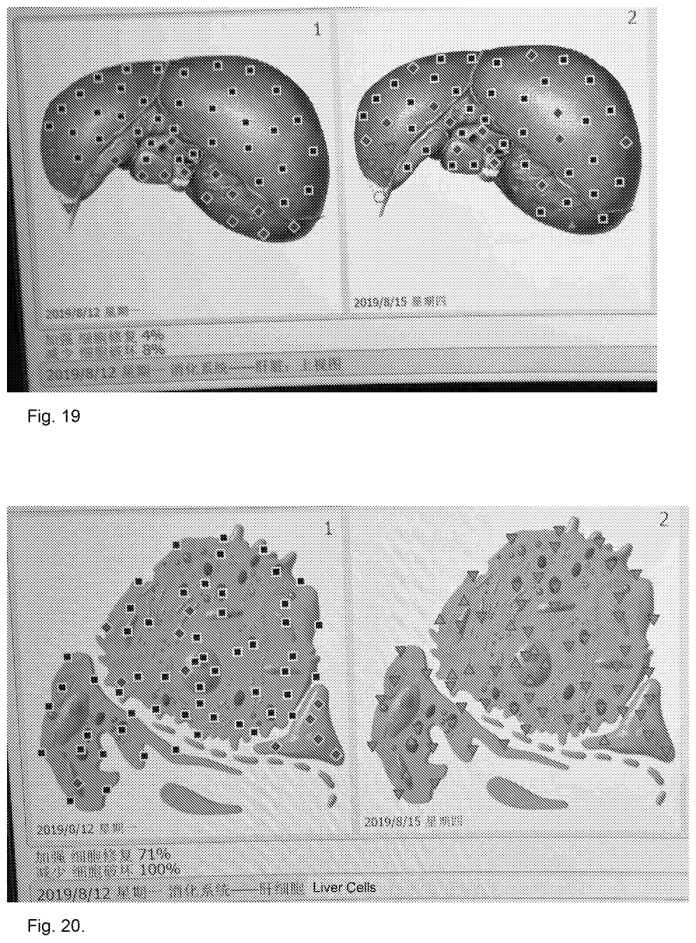

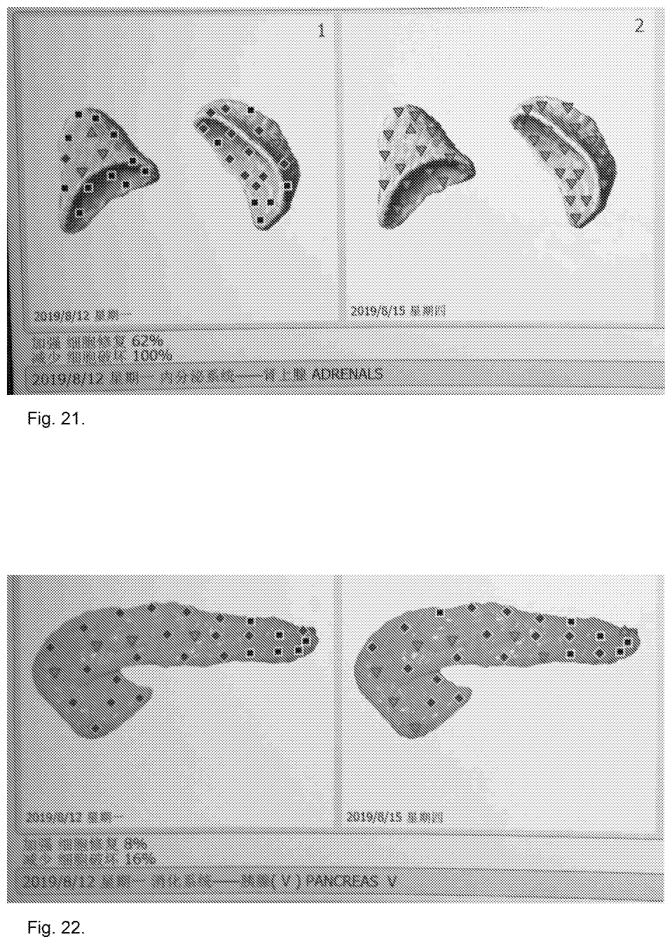

FIG. 19 is an image of the micromagnetic resonance imaging (mMRI) scan showing the improved cellular activities of the liver after the 63-year old man was treated inside of the scalar wave-powered healing bed for three nights as compared to the baseline condition. The cellular repair rate of the liver was increased by 4%, and the cellular destruction rate of the brain arteries was reduced by 8%. This result clearly indicated that the scalar wave treatment for three nights significantly improved the cellular activity of the liver. The liver is the critical organ for normal metabolism and for providing the whole body with energy, in addition to detoxifying the metabolic wastes. Liver improvement may need a longer period than just three days.

FIG. 20 is an image of the micro-magnetic resonance imaging (mMRI) scan showing the improved cellular activities of liver cells after the 63-year old man was treated inside of the scalar wave-powered healing bed for three nights as compared to the baseline condition. The cellular repair rate of the liver cells was increased by 71%, and the cellular destruction rate of the liver cells was reduced by 100%. This result clearly indicated that the scalar wave treatment for three nights significantly improved the cellular activity of the liver cells. The improved liver cell function would energize the body and benefit the overall health of the whole body every day.

FIG. 22 is an image of the micro-magnetic resonance imaging (mMRI) scan showing the improved cellular activities of adrenals after the 63-year old man was treated inside of the scalar wave-powered healing bed for three nights as compared to the baseline condition. The cellular repair rate of the brain arteries was increased by 62%, and the cellular destruction rate of the adrenals was reduced by 100%. This result clearly indicated that the scalar wave treatment for three nights significantly improved the cellular activity of the adrenals. A number of important hormones are produced inside of adrenals so the improved function of adrenals will make it easier to reach the hormone balance for the body.

FIG. 22 is an image of the micro-magnetic resonance imaging (mMRI) scan showing the improved cellular activities of the pancreas after the 63-year old man was treated inside of the scalar wave-powered healing bed for three nights as compared to the baseline condition. The cellular repair rate of the pancreas was increased by 8%, and the cellular destruction rate of the brain arteries was reduced by 16%. This result clearly indicated that the scalar wave treatment for three nights significantly improved the cellular activity of the pancreas. It might cause a better production of insulin and other digestive hormones produced inside of the pancreas when the pancreas becomes more active in cellular activities.

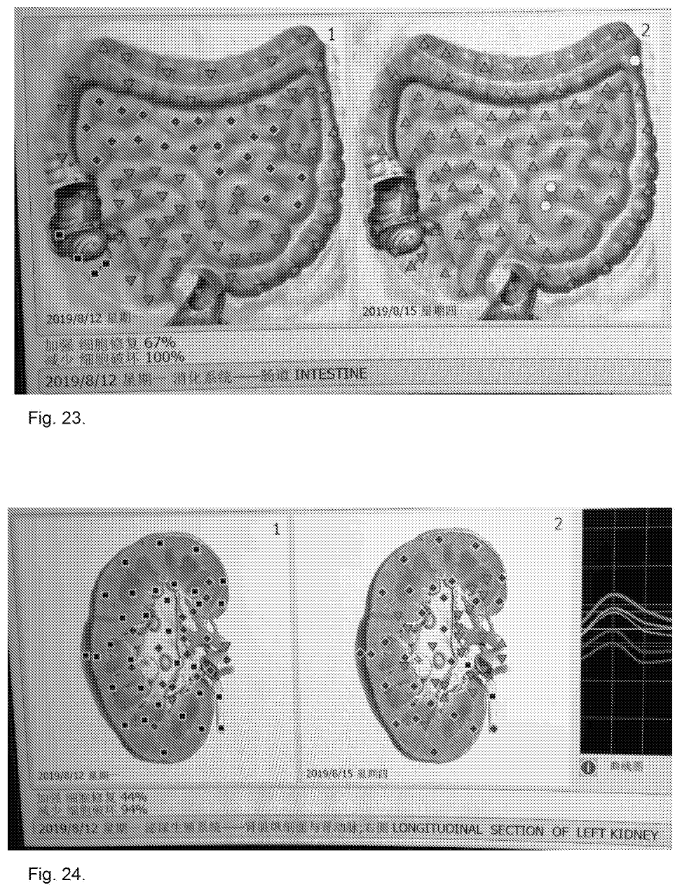

FIG. 23 is an image of the micro-magnetic resonance imaging (mMRI) scan showing the improved cellular activities of the intestine after the 63-year old man was treated inside of the scalar wave-powered healing bed for three nights as compared to the baseline condition. The cellular repair rate of the intestine was increased by 67%, and the cellular destruction rate of the brain arteries was reduced by 100%. This result clearly indicated that the scalar wave treatment for three nights significantly improved the cellular activity of the intestine. As reported by the patient, the treatment inside of the biocompatible scalar wave energy powered healing bed improved the function of the digestive tract of the patient and his energy level was higher than before.

FIG. 24 is an image of the micromagnetic resonance imaging (mMRI) scan showing the improved cellular activities of the kidney after the 63-year old man was treated inside of the scalar wave-powered healing bed for three nights as compared to the baseline condition. The cellular repair rate of the kidney was increased by 44%, and the cellular destruction rate of the brain arteries was reduced by 94%. This result clearly indicated that the scalar wave treatment for three nights significantly improved the cellular activity of the kidney. Improved the cellular activity of the kidney can benefit the body in numerous ways, particularly in timely removing the metabolic wastes from the body. The treatment inside of the biocompatible scalar wave energy powered healing bed improved the function of the urinary tract of the patient.

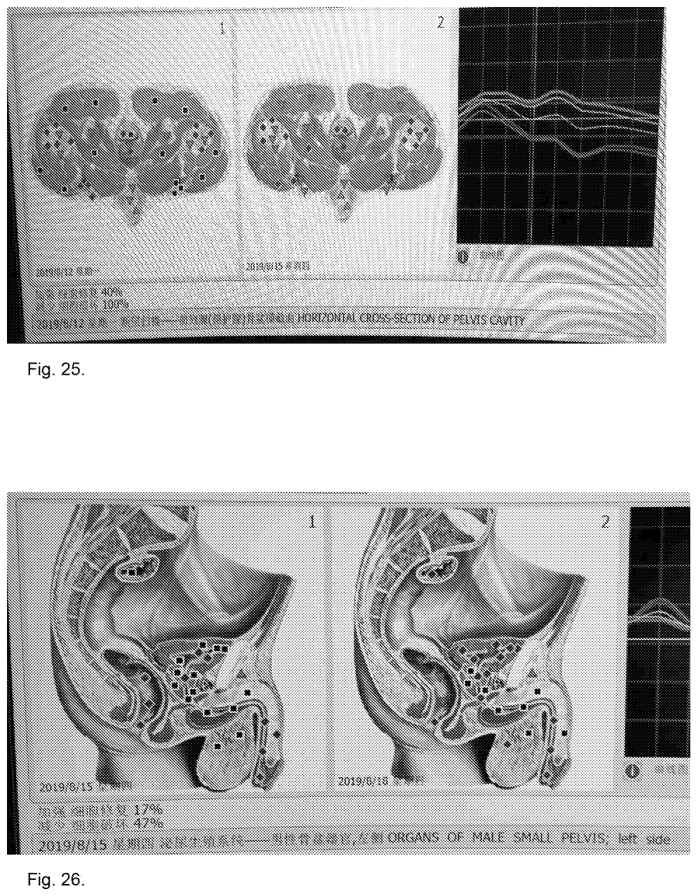

FIG. 25 is an image of the micro-magnetic resonance imaging (mMRI) scan showing the improved cellular activities of the pelvis cavity after the 63-year old man was treated inside of the scalar wave-powered healing bed for three nights as compared to the baseline condition. The cellular repair rate of the pelvis cavity was increased by 40%, and the cellular destruction rate of the brain arteries was reduced by 100%. This result clearly indicated that the scalar wave treatment for three nights significantly improved the cellular activity of the pelvic cavity.

FIG. 26 is an image of the micro-magnetic resonance imaging (mMRI) scan showing the improved cellular activities of male reproductive organs after the 63-year old man was treated inside of the scalar wave-powered healing bed for three nights as compared to the baseline condition. The cellular repair rate of the male reproductive organs was increased by 17%, and the cellular destruction rate of the brain arteries was reduced by 47%. This result clearly indicated that the scalar wave treatment for three nights significantly improved the cellular activity of the male reproductive organs.

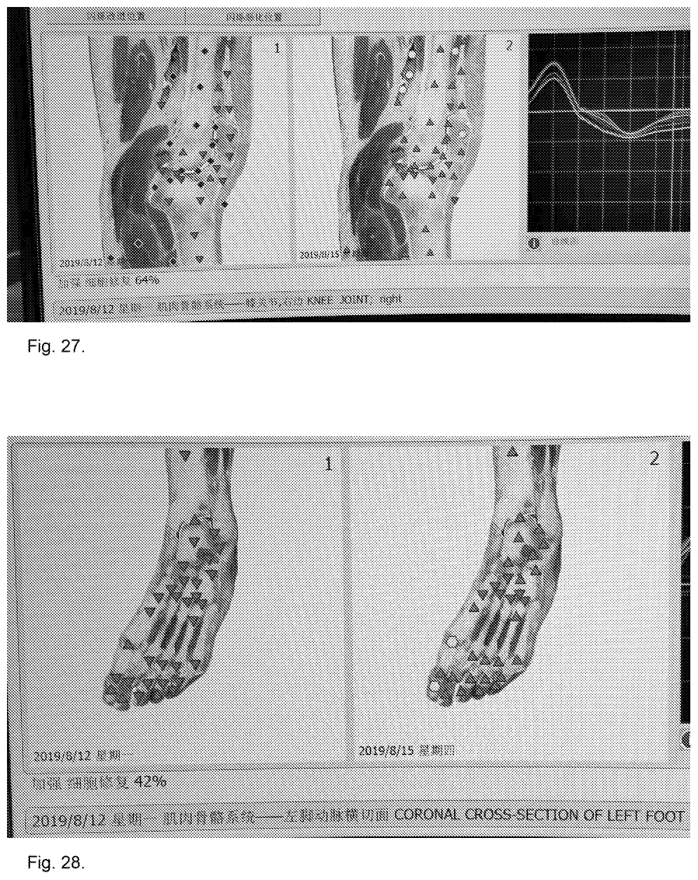

FIG. 27 is an image of the micro-magnetic resonance imaging (mMRI) scan showing the improved cellular activities of the knee joint after the 63-year old man was treated inside of the scalar wave-powered healing bed for three nights as compared to the baseline condition. The cellular repair rate of brain arteries was increased by 64%. This result clearly indicated that the scalar wave treatment for three nights significantly improved the cellular activity of the knee joint.

FIG. 28 is an image of the micro-magnetic resonance imaging (mMRI) scan showing the improved cellular activities of the left foot after the 63-year old man was treated inside of the scalar wave-powered healing bed for three nights as compared to the baseline condition. The cellular repair rate of the left foot was increased by 42%. This result clearly indicated that the scalar wave treatment for three nights significantly improved the cellular activity of the left foot.

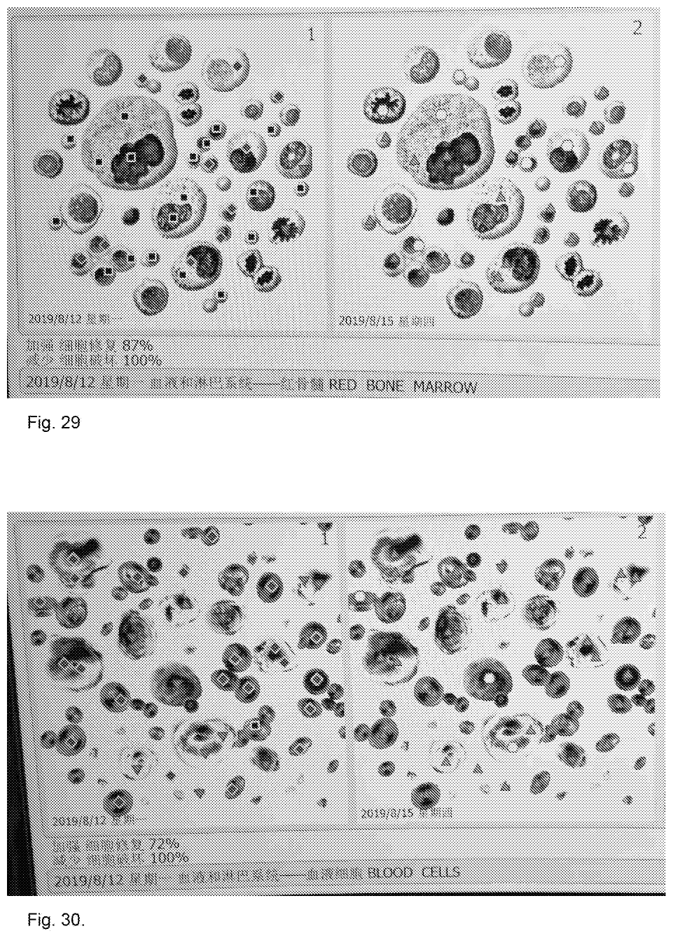

FIG. 29 is an image of the micro-magnetic resonance imaging (mMRI) scan showing the improved cellular activities of red bone marrow after the 63-year old man was treated inside of the scalar wave-powered healing bed for three nights as compared to the baseline condition. The cellular repair rate of the red bone marrow was increased by 87%, and the cellular destruction rate of the brain arteries was reduced by 100%. This result clearly indicated that the scalar wave treatment for three nights significantly improved the cellular activity of the red bone marrow.

FIG. 30 is an image of the micro-magnetic resonance imaging (mMRI) scan showing the improved cellular activities of blood cells after the 63-year old man was treated inside of the scalar wave-powered healing bed for three nights as compared to the baseline condition. The cellular repair rate of the blood cells was increased by 72%, and the cellular destruction rate of the brain arteries was reduced by 100%. This result clearly indicated that the scalar wave treatment for three nights significantly improved the cellular activity of the blood cells. This will help the body to have a better quality of the blood supply than that of the prior treatment.

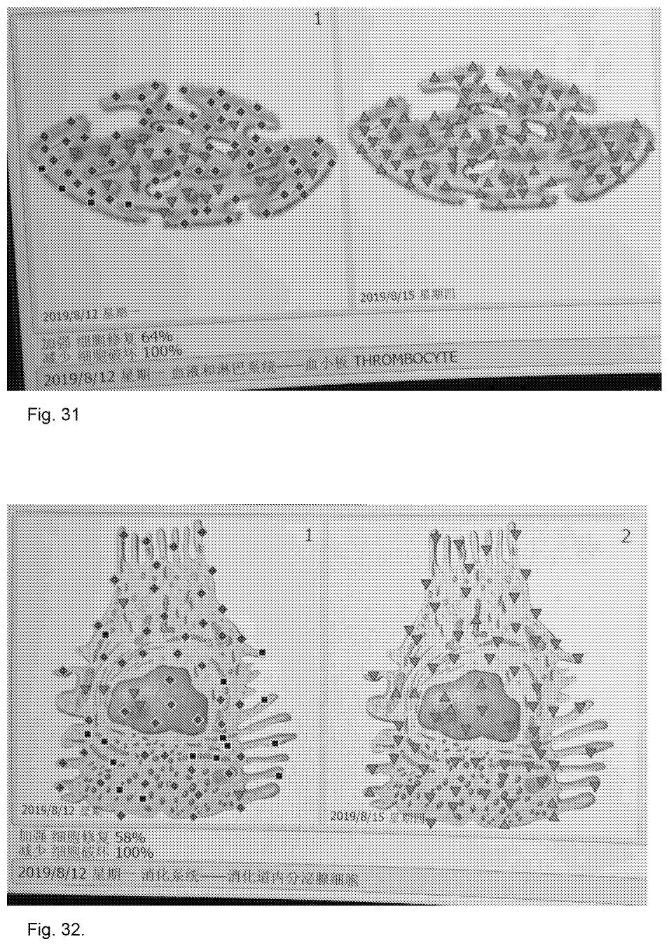

FIG. 31 is an image of the micro-magnetic resonance imaging (mMRI) scan showing the improved cellular activities of thrombocytes after the 63-year old man was treated inside of the scalar wave-powered healing bed for three nights as compared to the baseline condition. The cellular repair rate of the thrombocyte was increased by 64%, and the cellular destruction rate of the brain arteries was reduced by 100%. This result clearly indicated that the scalar wave treatment for three nights significantly improved the cellular activity of the thrombocyte. The healthier thrombocyte will help the body to have a normal blood fluidity and blood clot when needed.

FIG. 32 is an image of the micro-magnetic resonance imaging (mMRI) scan showing the improved cellular activities of digestive glands after the 63-year old man was treated inside of the scalar wave-powered healing bed for three nights as compared to the baseline condition. The cellular repair rate of the brain arteries was increased by 58%, and the cellular destruction rate of the digestive glands was reduced by 100%. This result clearly indicated that the scalar wave treatment for three nights significantly improved the cellular activity of the digestive glands. The treatment inside of the biocompatible scalar wave energy powered healing bed improved the function of the digestive system of the patient.

FIG. 33 is an image of the micro-magnetic resonance imaging (mMRI) scan showing the improved cellular activities of the lymphatic vessel after the 63-year old man was treated inside of the scalar wave-powered healing bed for three nights as compared to the baseline condition. The cellular repair rate of the lymphatic vessel was increased by 9%, and the cellular destruction rate of the brain arteries was reduced by 17%. This result clearly indicated that the scalar wave treatment for three nights significantly improved the cellular activity of lymphatic vessels.

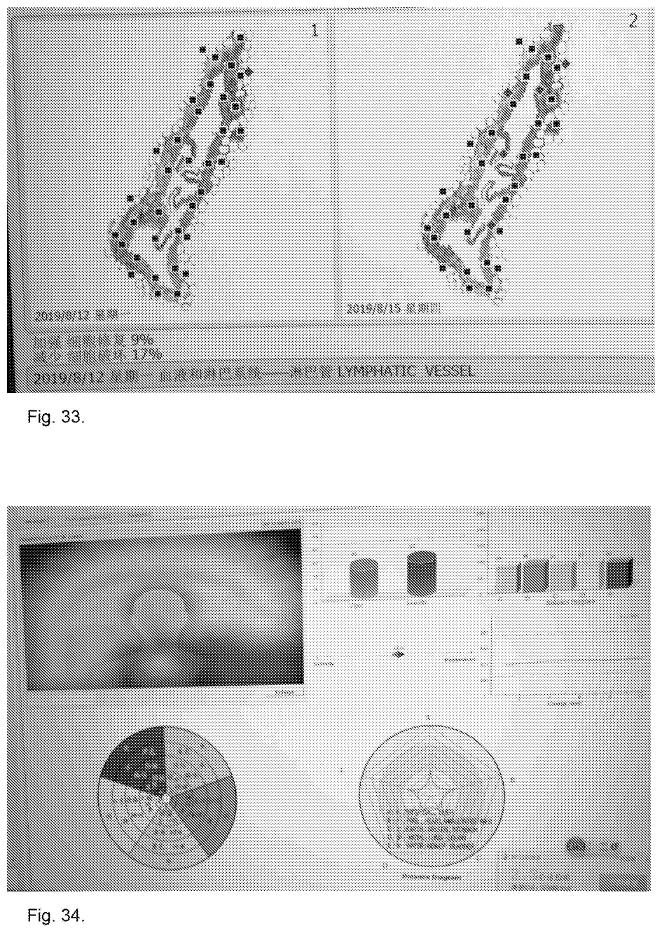

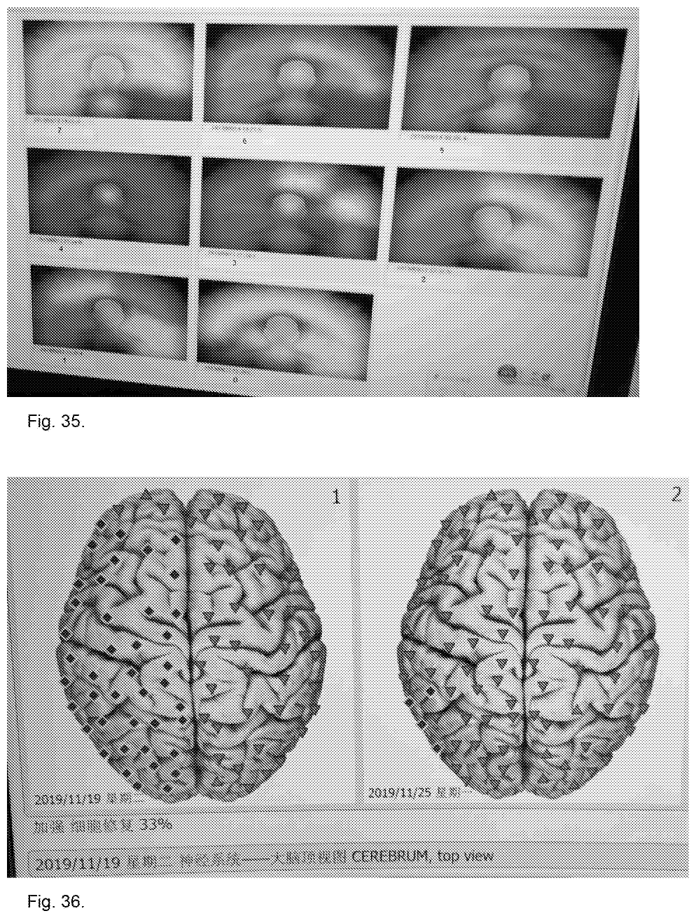

FIG. 34 is an image of the five-character scanned using a Traditional Chinese Medicine diagnostic instrument, which was showing the improved five-characters after the 63-year old man was treated inside of the scalar wave-powered healing bed for three nights as compared to the baseline condition. The five characters were very well balanced. The Yin side was slightly higher than the Yang side due to the common phenomenon that the aged individual tends to have a higher Yin and lower Yang. This result clearly indicated that the scalar wave treatment for three nights significantly improved the balance of Yin and Yang, as well as the fiver critical characters.

FIG. 35 is an image of the life-energy scanning using a Traditional Chinese Medicine diagnostic instrument. It is showing that the life-energy was gradually increased from the baseline (0), to after one-night sleep (1-2), to after two-nights sleep (3-4), and then to after three-night sleep (5-7) inside of the scalar wave-powered healing bed. After being treated for three nights inside of the scalar wave energized healing bed, the man significantly improved his life energy every day.

FIG. 35 is an image of the micro-magnetic resonance imaging (mMRI) scan showing the improved brain cellular activities of the cerebrum (top view) after the 50-years old female stroke-paralyzed patient was treated inside of the scalar wave-powered healing bed for six days-nights as compared to the baseline condition. The cellular repair rate of brain arteries was increased by 33%. Because her left side of the brain was damaged during the acute stroke phase, the recovery of the left side of the brain was so special for this stroke patient. This test result clearly indicated that the scalar wave energy treatment for six nights significantly improved the cellular activity of the brain (cerebrum), which was very well correlated with the motion recovery of the right arm and right leg after one-week treatment inside of the biocompatible scalar wave energy powered healing bed.

FIG. 37 is an image of the micro-magnetic resonance imaging (mMRI) scan showing the improved cellular activities of nuclei of cranial nerves after the stroke-paralyzed patient was treated inside of the scalar wave-powered healing bed for six days-nights as compared to the baseline condition. The cellular repair rate of the brain tissue was increased by 27%, and the cellular destruction rate of the brain arteries was reduced by 100%. This result clearly indicated that the scalar wave treatment for three nights significantly improved the cellular activity of the brain tissue, which was very well correlated with the motion recovery of the right arm and right leg after one-week treatment inside of the healing bed.

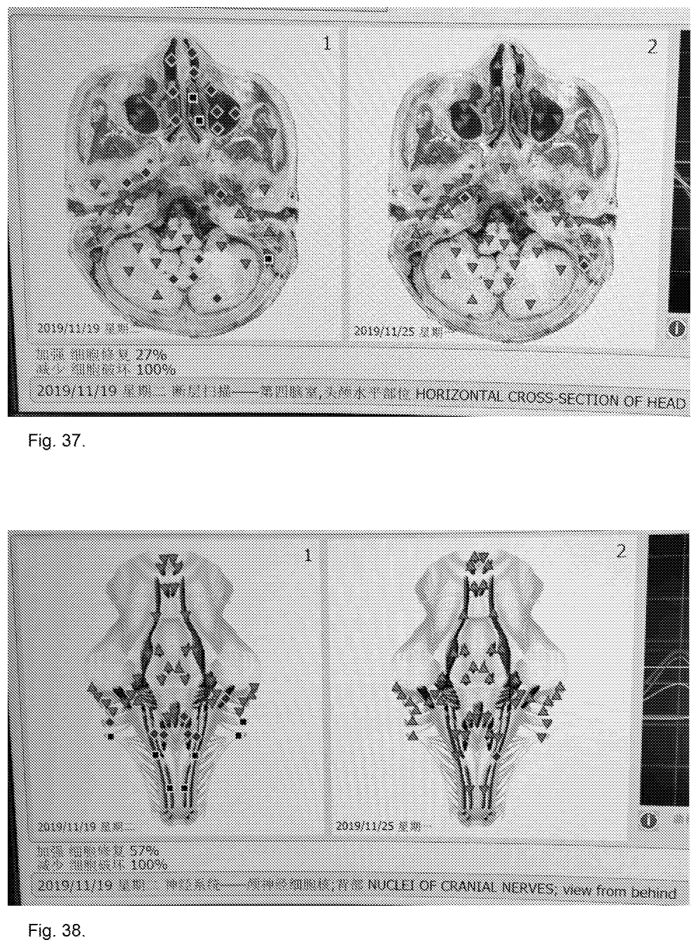

FIG. 38 is an image of the micro-magnetic resonance imaging (mMRI) scan showing the improved cellular activities of nuclei of cranial nerves after the stroke-paralyzed patient was treated inside of the scalar wave-powered healing bed for six days-nights as compared to the baseline condition. The cellular repair rate of the nuclei of cranial nerves was increased by 57%, and the cellular destruction rate of the brain arteries was reduced by 100%. This result clearly indicated that the scalar wave treatment for three nights significantly improved the cellular activity of the nuclei of cranial nerves.

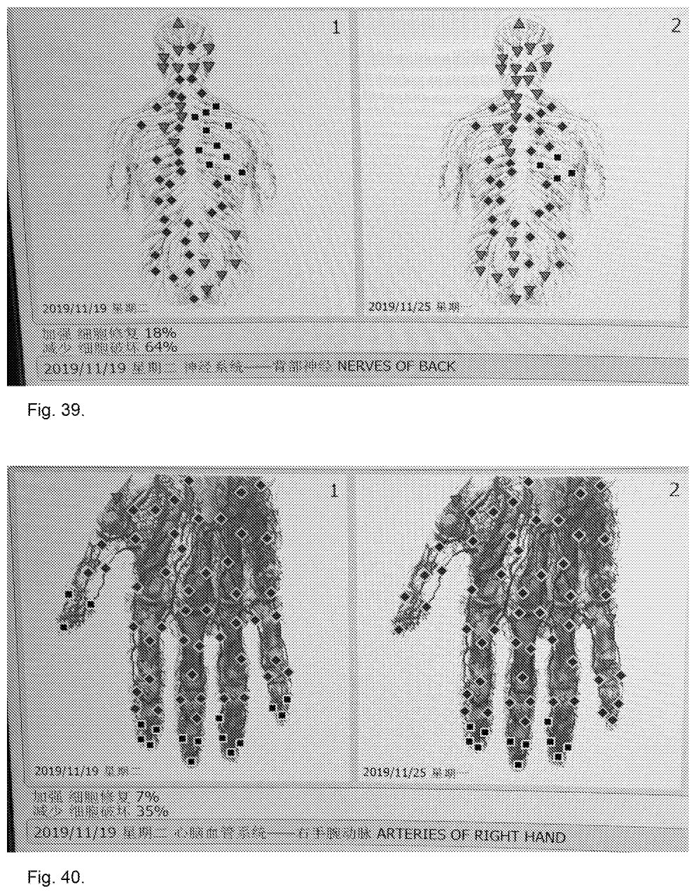

FIG. 39 is an image of the micro-magnetic resonance imaging (mMRI) scan showing the improved cellular activities of the back-nerve network after the stroke-paralyzed patient was treated inside of the scalar wave-powered healing bed for six days-nights as compared to the baseline condition. The cellular repair rate of the brain arteries was increased by 18%, and the cellular destruction rate of the back-nerve network was reduced by 64%. This result clearly indicated that the scalar wave treatment for three nights significantly improved the cellular activity of the back-nerve network, which was very well correlated with the motion recovery of the right arm and right leg after one-week treatment inside of the healing bed.

FIG. 39 is an image of the micro-magnetic resonance imaging (mMRI) scan showing the improved cellular activities of the right hand after the stroke-paralyzed patient was treated inside of the scalar wave-powered healing bed for six days-nights as compared to the baseline condition. The cellular repair rate of the right hand was increased by 7%, and the cellular destruction rate of the right hand was reduced by 35%. This result clearly indicated that the scalar wave treatment for three nights significantly improved the cellular activity of the right hand, which was very well correlated with the motion recovery of the right arm and right hand after one-week treatment inside of the healing bed.

The bio-recharge energy system disclosed in the current invention increased the cellular repair rate of all types of organs and tissues, and the cellular destruction rate of all types of organs and tissues were significantly decreased, from the head to feet. The invented scalar wave generator and the other parts have formed a very unique biological energy field to empower all cells, all tissues, all organs, and the entire body. Therefore, the patient can sleep better, can have much higher clarity of the brainpower, and a much better health status overall.

The new therapeutic effects of the specially generated scalar wave field were used to treat 7 stroke-paralyzed patients. They all improved the motion ability very meaningfully. All 7 patients became life independent. The newly generated special energy field is verified to be beneficial to a man, a woman, a young, an old, and anyone in between. These therapeutic effects are important parts of the claims of the current invention. This invention has a very broad therapeutic effect and a high level of safety. No side effects were observed from any users.

The bio-recharge system can be called a quantum cell activator. After the use-tested of the current invention, the inventors observed numerous excellent effects from the 600 person-times in three years. The quantum-cell activator provided by the present invention did not only restore the users' energy level but also improved the sub-health constitution. More impressionably, the patients with high blood pressure, diabetes, terminal cancer, insomnia, depression, heart failure, and osteoporosis, recovered to a very satisfactory level. Many users' physical and mental exhaustion was significantly improved, which were also parts of the therapeutic effects. Those elderly who were physically weak and unable to take care of themselves were significantly improved and could self-support for daily life. The below examples are to summarize the therapeutic effects experienced by the 600 patients.

### EXAMPLES

### Example 1. Chronic Fatigue Syndrome

During the 3-year user-test period, we treated 130 patients suffering from chronic fatigue syndrome. They had physical and mental exhaustion, some with difficulty walking. After treated with the invented device for at least two nights or more times, nearly 90 patients had significant recovery in physical strength, and those symptoms of the steps brisk and fatigue were eliminated. The mood became pleasant; the memory was improved, and they felt 5 to 10 years younger than their current age for those who slept inside of the invented device for longer than one month.

### Example 2. Insomnia

More than 90 insomniac individuals were treated with the invented device. The 70 patients were significantly improved, and it was easy to fall asleep, and the deep sleep time was prolonged. Among them, they had insomnia for more than 10 years, some even decades. Patients who failed multiple treatments with modern medicines were treated with the invented device for two to three months, and their quality of sleep was significantly improved, so they could have a deep sleep.

### Example 3. Common Colds

More than 150 middle-aged and elderly people with chronic diseases have been treated with the invented device, and 110 people have significantly improved their quality of life, and enhanced immunity. They were no longer susceptible to colds. Even if someone had a cold, they could recover much fast. Among them, the 50 elderly regained their physical strength and could live freely.

### Example 4. High Blood Pressure

The 30 patients with high blood pressure did not need antihypertensive drugs after the therapy. Two of those patients were a 51 years old male and his wife. They both had hypertension. They installed two sets of the invented device in early January 2017. Since then, as long as they were not on a business trip, he and his wife were sleeping inside of the invented device as much as possible. After the nightly treatment for 4 weeks, plus exercise and a vegetarian diet, their blood pressure was normalized. His shoulders, knees, and wrists had recovered completely after the abnormality for 20 years. Now, although he was in his fifties, his body shape and weight were the same in his early 30s. His wife's health was also improved significantly. Her body size has also returned to the age of thirty. Her height is 164 cm and her weight 60 kg. They got up at about 5 o'clock every morning, energetic, full of energy, youth face, and moisturizing. There is a tendency to reverse aging for both of them after long-term nightly sleeping inside of the invented device. He wrote to the invented device R&D Team staff in December 2017 that “Thanks to the R&D team to provide the invented device, thank you for the guidance and help of all the noble people in your life so that we both husband and wife can have such a good status of health. Very much appreciated!”

### Example 5. Diabetes

The 19 people with diabetes did not rely on insulin injections. One of them was a female physician with type 2 diabetes for two years. The fasting blood sugar was between 10 and 11, and her physical strength was almost all gone. She was treated inside of the Healing Bed in August 2015 for 23 weeks. Her fasting blood glucose was controlled between 6.1 and 6.4. She was not afraid of eating anything, and she was energetic and could help her daughter to take care of the grand-children.

### Example 6. Misshape due to Osteoporosis

Ten middle-aged people suffered from osteoporosis and had a bad spinal column. Surprisingly, the 10 people gained the body height of about 1 cm after the invented device treatment for about 1 to 2 months. This was most likely due to the straightened backbone.

### Example 7. Breast Cancer

The two patients with stage 4 breast cancer were treated with the invented device. The first patient with breast and then cervical cancer, non-treatable with any chemotherapy. The second breast cancer patient was also failed with the chemotherapy. After each of them was treated with the invented device for 4 to 8 weeks, they were out of the dangerous phase and entered the rehabilitation period. Both of them are living after one or two years of treatment, respectively.

### Example 8. Colon Cancer

A male patient with stage 4 colon cancer was treated with the invented device periodically. When the patient's condition was improved to have a good level of energy, the patient went a free-living life. When the patient felt tired, he went back to the treatment center for one more month. This in and out was repeated for the last two years. The patient is living normally.

### Example 9. Lung Cancer

A female patient with stage 4 lung cancer was treated with the invented device for four weeks and improved her overall health status significantly. Her family installed a healing bed in their house so the patient can be continually treated. The patient has been a free-living person since then.

### Example 10. Gastric Cancer

A 53-year old male patient with stage 4 cardia cancer was treated with the healing bed. His cancer spread up and down along the digest cavity, and the stomach and esophagus were invaded by cancer after eight courses of chemotherapy. His face was very dark and his overall health condition was very bad before the treatment. After entering into the healing bed for ten days, his appetite was improved significantly and he often asked for food. His face was changed to a much healthier looking. After the 4-weeks treatment, the patient was examined in a hospital and his cancer volume was smaller than before. After continually being treated for another 10 months, the patient was able to have his normal life. These cancer bio-marks became negative.

### Example 11. Leukemia

A 70-year old female patient had stage 4 leukemia. Her bone marrow activities were very poor and anemia was very severe after many courses of chemotherapy. Her blood type was an RH negative and very hard to get a blood supply. The hospital managed to give her a blood transfusion every 4 days. After the last blood transfusion, the hospital asked the patient to go home to wait for the end of her life. After this patient was treated inside of the healing bed for 2 weeks, her anemia sign was almost gone without any more blood transfusion. The patient had a good level of energy and could talk with her family members easily. The patient is still living after starting the treatment inside the healing bed for 6 months.

### Example 12. Thyroid Cancer along with Uterus Cancer

A female patient had surgery to remove her thyroid cancer and uterus cancer. Since August 2015, this patient with a bodyweight of 30 kilograms began to use the invented healing bed. The body constitution was gradually improved. The whole-body joints were not as painful and inflated as before the treatment. Her hands began to able to hold things, her sleep was improved, and the appetite was increased. She gained a weight of 9 kilograms, and the legs and feet became easy in walking. By the end of the treatment for two months, her walk speed was very fast, even faster than the common younger people who were walking in the same area. She did not catch a cold since then. In the past, if she laid down and forget to cover herself for a few minutes, she would catch a cold. Now, she was much strong against the cold. Her height gained by 10 mm, because she was full of energy, and her chest and back were straight. Her bones and joints and ligaments were restored. Her vision was enhanced, and she even did not need the reading glasses. Her memory was also enhanced. She could remember a phone number after reading once. She could send text messages. Her physical fitness was improved and did not feel tired after working at home for 10 hours a day.

### Example 13. Enhancing Immunity.

More than 60 children and adolescents were treated with the invented device and they generally grow well, not easy to get sick, and their memory was enhanced.

### Example 14. Gynecological Dysmenorrhea

After treated for two or three nights inside of the invented device, more than 30 women had no more painful menstruation. Someone had an uncomfortable feeling, particularly backache during the period before treatment. After the treatment, the symptoms were gone. Four women had a recurrence of the period after menopause for a year or so.

### Example 15. Prostatitis

The 80 male patients with prostatitis had a high frequency of night urination. After treated with the invented device for two to three nights, their conditions were improved significantly. More than 50 people reduced the number of night urination to 1 time.

### Example 16. Depression

Thirty (30) people were depressed before the treatment. They were irritated, some even did not want to live. After conditioning for two or three months inside of the invented device, 20 people were happy and cheerful, and their attitude towards life was positive.

### Example 17. Radiations

The invented device was used to help the 6 patients who were harmed by various electromagnetic radiation.

### Example 18. Viral Infection

The invented device was used to help the 8 patients to eliminate the viral infection, and to reduce or eliminate the pain caused by the virus infection.

### Example 19. Male Function Disorder

Among those 130 patients who suffered from chronic fatigue syndrome, 72 were male adults. They had a certain degree of erectile dysfunction before the treatment. After treated with the invented device for 3 or more nights, a supermajority of these men improved their libido which was reflected by the reappearance of the morning wood phenomenon.

### Example 20. Mental Health Disorder

As experienced by the majority of the 600 treated patients, the Healing Bed made with the innovative technology can increase consciousness, improved emotions, and could quickly enter a state of calm and peace from the arrogant disorder. It helped to achieve a harmonious, pleasant, and balanced high-frequency state of the body, mind, and spirit; to activate and balance the body and mind. It also increased intuition and enhances concentration.

### Example 21. Spinal Disc Degeneration

About one-third of the 600 patients had a different level of the degenerated spinal disc, and neck-back pain was common among them. Sleeping inside of the bed full of the scalar wave energy improved their symptoms of the degenerated disc. Scalar energy can recharge the cells in the disc, and give the cells much-needed energy. Once the cells are fully recharged, the disc will plump up and return to healthy and well-hydrated. Scalar energy can help improve blood circulation by promoting the permeability of cell walls, which improves the intake of nutrients and water into the disc cells, keeping them hydrated. Additionally, scalar energy can relieve inflammation and pain by facilitating the flow of blood to the spine.

### Example 22. Cerebral Infarction (Stroke-Related Paralysis)

Seven patients had a stroke-related paralysis. Before the treatment with the current invention, their one-sided limb was unable to move and lost function. After treated inside the healing bed for 2 to 45 days, the seven patients all recovered to a satisfactory degree since each of them responded to the treatment with a different sensitivity. They can freely walk around, and their hands can do common jobs such as eating and dressing. Their intelligence level was also improved, so did their mental health.

### Example 23. Myocardial Infarction

A male patient was examined in a hospital and reported that his heart blood vessels were 85% blocked, and he often had angina. In June 2016, he was treated inside of the invented device. During these 40 days, the number of anginas was gradually reduced, and the duration of angina was gradually shortened, and the uncomfortable feeling of the heart was eliminated. This 40-day treatment inside of the invented device was very effective.

### Example 24. Weak Heart

A female patient engaged in medical therapy for many years. Her ad a long-term history of using an instrument to treat people with electrotherapy. During her professional work, she delivered the weak electric currents to the patients through her own hands to dredge the patient's meridians and blood vessels. The long-term work leads to having an extreme weakness. Her heart was damaged and she had angina. In November 2015 she started to be treated inside of the invented device for 2 months. Her heart discomfort gradually disappeared, and her heart function was substantially restored.

### Example 25. Dependence on Walking Sticks

Ten elderly people were relying on a walking stick for a walk before the treatment. After being treated for at least 5 nights inside of the invented device, the 10 elderly people did not need their walking sticks. Their daily life was much happier.

### Example 26. Male Prostate Enlargement

Two men had prostate enlargement and had frequent urination at night and during the day time. After treated inside of the invented device for two nights, the number of urination during the night was significantly reduced. They could sleep longer. After treated for one week, they only need to go to the bathroom one time or none.

### Example 27. Reflux Esophagitis

A female patient suffered from reflux esophagitis for years. In the past, the esophagus always had upwards of acid water. The doctor diagnosed her to have reflux esophagitis. After treated inside of the Healing Bed 5 times, her condition was improved greatly and she stopped having acid water.

### Example 28. Live Function Disorder

A man felt that his liver area was not comfortable during the past few months. On Jun. 28, 2017, he checked in the local hospital. Alanine aminotransferase was 75.9 (reference range 9.0˜50) and aspartate aminotransferase was 58.6 (reference range 15˜40). He found a famous Traditional Chinese medicine (TCM) practitioner locally and took 15 TCM formulas. One month later, on July 14, he went back to the same local hospital for another examination. The alanine aminotransferase was 86.2 (reference range 9.0˜50), Aspartate aminotransferase was 51.1 (reference range 15˜40). Alanine aminotransferase did not decrease but increased. He took another 15 TCM formulas. On August 4th, he went to the same hospital for the 3rd examination. Alanine aminotransferase was 103.1 (reference range 9.0˜50) and aspartate aminotransferase was 63.7 (reference range 15˜40). The two transaminase enzymes did not decrease but increased. On Aug. 22, 2017. He was to be treated inside of the invented device. At the very beginning, he experienced more uncomfortable in the liver area, his stomach was swollen, he didn't want to eat. After 10 days of the treatment, his stomach was no more swollen, and he wanted to eat. He went back to the same hospital on Sep. 2, 2017, for the fourth laboratory test. He was really happy to read the new test results. Alanine aminotransferase 44.8 (reference range 9.0˜50), aspartate aminotransferase 34.7 (reference range 15˜40). The invented device treatment normalized his live enzymes.

### Example 29. Traumatic Wound Healing

A male patient was injured in the calf when he participated in the construction of a big building in 1959. For the last 50 years, he went through various treatments, the skin was dark and he was not cured. The symptoms were like vasculitis. In June of 2017, his legs were squatting again, and the wound was infected. It was not sealed. After being treated inside of the invented device for 3 weeks, his wound in the leg slowly healed and the surface was now flat.

### Example 30. Severe Pain

Case 1. A man had painful and dying pain in the right liver area for several years. His family has a history of liver disease. In September 2016, he was treated with the invented device. After treated for 40 days, his hepatic pain disappeared.

Case 2. A lady had inflammation of the ligament fascia behind her knee for several years. It is a painful sensation. After treated inside of the invented device for 1 week, she did feel pain anymore.

Case 3. A female patient was a retired officer. She had pain everywhere in her body. Since she was treated inside of the invented device for one week, her pain level was reduced very significantly. Two weeks thereafter, she did not feel much pain at all.

### Example 31. Digestive Diseases

A female patient used to have spleen and stomach disharmony, flatulence, indigestion, thin stool, and poor absorption. After being treated inside of the invented device for one week, the function of the digestive system was improved, and the stool was in a normal shape. The amount of food intake has increased.

### Example 32. Breathing Problem due to Nasal Polyps

A boy, a fifth-grade student, suffered from poor breathing due to nasal polyps, adenoid hypertrophy, and had a serious sleep disorder. The eye-cycles were black, his face was yellow, his growth not normal, and his concentration in the classroom was not there. Since the treatment inside of the Tesla Healing Bed in September 2017, the breathing became smooth, the sleep state was improved, and the concentration of attention was much better. His academic performance was up and gained the height back to normal.

### Example 33. Beauty, Youth Looking Effect

It was observed that almost everyone who has been in the Healing Bed for more than 7 nights had the experience that the skin is soft and fine, smooth and firm, and the eyelids that had been pulled down were retracted, and some of them turn back into the double eyelids. They looked young and rejuvenated. Two females had the most obvious changes since they used the healing bed for more than 2 months.

### Example 34. Constipation

A male patient had five days of no stool due to constipation. He was suffering so much and rolled himself on the ground with sweat. His family member brought him into the Healing Bed and drank a vegetable soup. Two hours late, the stool was out. He was liberated from his severe constipation.

### Example 35. Gynecological Diseases

Case 1. A female had a blocked fallopian tube and was uncomfortable during menstruation. In June 2016, she was treated with a Healing Bed for 2 weeks. Then, the fallopian tube becomes smooth, and the menstruation was normal. She felt that she was younger than herself five years ago.

Case 2. A lady had an abnormal menstrual period, pain during menstruation, and poor blood color. After being treated inside of the invented device for 3 weeks, her symptoms were gone. She had a normal life.

Case 3. A young lady used to have stomachache and backache during the menstrual period. After slept inside of the invented device for 3 months, she did not feel pain during menstruation and dared to drink the cold liquids.

### Example 36. Enhance Intuition, Improve Creativity and Inspiration

A man and an unrelated woman had the same experience of enhancing their intuition, improving creativity and inspiration after treated inside of the healing bed for three or more nights. They then wanted to solve some puzzles in their hearts without paper or pen. After sleeping on the invented device, they wake up in the morning to have inspiration and a clearer mind and could resolve very complicated problems easily.

### Example 37. Hyperthyroidism

An adult woman had hyperthyroidism. She felt the all-powerless, irritated, anxious. On Oct. 1, 2016), she entered into the invented device and slept there for 4 days. She had a feeling of physical fitness, without being irritated.

### Example 38. Depression or Anxiety:

Two adult females used to have depression and anxiety. They felt that their life had no meaning and was very boring. They didn't want to live. After sleeping in the invented device three times, they became better off, could eat and sleep, and lived happily.

### Example 39. Back Injury

A female was doing garment processing and was injured in 2017. The doctor said that she must take a rest for three months. She then slept in the invented device for two weeks. She felt that her back pain due to injury was reduced and her skin was getting better. Finally, her sleep was getting better, stepping on the stairs of the high building was very easy, and she even was running with the small steps when was running upstairs.

### Example 40. Vision Improvement

An adult man had aging eyes and blurred visions. After sleeping inside of the invented device for two weeks, his vision was restored to a satisfactory degree.

We claim:

1. A biocompatible scalar wave energy generation system to increase stem cell production, to promote cell growth, to facilitate cell repair, to reduce cell damage, to improve health, and to treat a disease of a mammal, comprising:
a stationary or a circulatory scalar wave energy filed generator, comprising a bracket and a supporting frame system, a scalar wave energy field source component, a scalar wave energy field conduction system, a scalar wave energy field flow-guide controller, a scalar wave energy field collector, and a scalar wave energy field release device to enable the scalar wave energy to flow around to a mammal in a stationary or a circulatory manner; and
a rest apparatus, like a bed, or a pad, or a chair, empowered with scalar wave energy to enable a mammal to receive stationary and/or circulatory scalar wave energy so the entire body of a mammal can increase the bioenergy potential.

2. The mammal referred to in claim 1 is a human or a domestic animal.

3. The disease or disorder referred to in claim 1 is a brain disease or disorder, such as stroke, or brain injury.

4. The disease or disorder referred to in claim 1 is a respiratory disease or disorder, such as asthma, or COPD.

5. The disease or disorder referred to in claim 1 is cardiovascular disease or disorder, such as hypertension.

6. The disease or disorder referred to in claim 1 is a digestive system disease or disorder, such as Crohn's disease.

7. The disease or disorder referred to in claim 1 is a liver disease or disorder, such as chronic hepatitis.

8. The disease or disorder referred to in claim 1 is a renal system disease or disorder, such as chronic kidney inflammation.

9. The disease or disorder referred to in claim 1 is a reproductive disease or disorder, such as erectile dysfunction or dysmenorrhea.

10. The disease or disorder referred to in claim 1 is an immunity disease or disorder, such as autoimmune disease, or Lyme disease.

11. The disease or disorder referred to in claim 1 is an endocrine system disease or disorder, such as diabetes.

12. The disease or disorder referred to in claim 1 is a nervous system disease or disorder, such as multiple sclerosis.

13. The disease or disorder referred to in claim 1 is a circulatory system disease or disorder, such as anemia.

14. The disease or disorder referred to in claim 1 is a malignant disease or disorder, such as lung cancer.

15. The disease or disorder referred to in claim 1 is a skeletal-muscle disease or disorder, such as arthritis.

16. The disease or disorder referred to in claim 1 is a psychological disease or disorder, such as depression.

17. The disease or disorder referred to in claim 1 is a degenerative or aging disease or disorder, such as Alzheimer's disease, or dementia.

18. The disease or disorder referred to in claim 1 is an infectious disease or disorder, such as the hepatitis C virus infection.

19. A process of using a therapeutic device generating and releasing bioenergy from a stationary scalar wave energy field generator or a circulatory scalar wave energy filed generator, to enable the entire body of a mammal to increase the energy potential, to promote stem cell production, to promote cell growth, to facilitate cell repair, and to reduce cell destruction, to cause the cells, organs, systems and the entire body of a mammal become healthy, and to treat a disease or a disorder of a mammal.

20. A method of using a device generating and releasing bioenergy from a stationary scalar wave energy field generator or a circulatory scalar wave energy filed generator, to enable the entire body of a mammal to increase the energy potential, to promote stem cell production, to promote cell growth, to facilitate cell repair, and to reduce cell destruction, to cause the cells, organs, systems and the entire body of a mammal become healthy, and to treat a disease or a disorder of a mammal.
